SEQUENCE LISTING (2 sequences)

SEQ ID NO: 1 - DNA, 20 bp, synthetic construct
  source 1..20: mol_type = other DNA
tttttttttgtttttttttt

SEQ ID NO: 2 - DNA, 60 bp, synthetic construct
  source 1..60: mol_type = other DNA
tttttttttgtttttttttgtttttttttgtttttttttgtttttttttgtttttttttt

(19) United States
(12) Patent Application Publication
(10) Pub. No.: US 2025/0059528 A1
(43) Pub. Date: Feb. 20, 2025

(54) LIGANDS AND METHODS OF MAKING LIGANDS FOR AFFINITY CAPTURE ON A SURFACE OF MRNA IN A SOLUTION

(71) Applicants: Georges Belfort, North Falmouth, MA (US); Thomas Neuman, Fort Lauderdale, FL (US); Riddhi Banik, Troy, NY (US); Todd Przybycien, Milton, VT (US); RENSSELAER POLYECHNIC INSTITUTE, Troy, NY (US)

(72) Inventors: Georges BELFORT, North Falmouth, MA (US); Mirco Sorci, Troy, NY (US); Thomas Meuman, Fort Lauderdale, FL (US); Riddhi Banik, Troy, NY (US); Todd Przybycien, Milton, VT (US); Lionella Brzybycien, Milton, VT (US)

(73) Assignee: RENSSELAER POLYECHNIC INSTITUTE, Troy, NY (US)

(21) Appl. No.: 18/936,186

(22) Filed: Nov. 4, 2024

Related U.S. Application Data
(63) Continuation of application No. PCT/US2023/069964, filed on Jul. 11, 2023.
(60) Provisional application No. 63/388,158, filed on Jul. 11, 2022.
(60) Provisional application No. 63/423,267, filed on Nov. 7, 2022.
(60) Provisional application No. 63/452,590, filed on Mar. 16, 2023.
(60) Provisional application No. 63/469,680, filed on May 30, 2023.
(60) Provisional application No. 63/469,924, filed on May 31, 2023.
(60) Provisional application No. 63/715,364, filed on Nov. 1, 2024.
(60) Provisional application No. 63/547,242, filed on Nov. 3, 2023.

(51) Int. Cl. C12N 15/10; C07K 14/00; C07K 17/06

(52) U.S. Cl. CPC C12N 15/1006; C07K 14/003; C07K 17/06

(57) ABSTRACT

Separation constructs such as membranes, porous beads, etc. are modified with a plurality of oligomeric ligands. The ligands are bound to the surface of the separation substrates via linker constructs such as acrylate groups or azide groups, e.g., via Single-Electron Transfer-Living Radical Polymerization (SET-LRP). A plurality of spacer constructs, such as polyethyleneglycol (PEG) groups and hydrocarbyl groups, separate the linker constructs from oligomer constructs. The oligomer constructs can include between 5% and about 10% guanine and about 90% to about 95% thymine, and can further include between about 20 and about 60 nucleotides including at least 15 thymines and at least 1 guanine. The oligomer constructs exhibit improved binding of mRNA with oligo-dAₙ tails, e.g., for purification of mRNA production and commercialization, enabling fast, efficient, and continuous production of mRNA vaccines such as those against coronaviruses, e.g., SARS-CoV-2.

### CROSS REFERENCE TO RELATED APPLICATION(S)

This application is a continuation of International Patent Application No. PCT/US23/69964, filed Jul. 11, 2023, which claims the benefit of U.S. Provisional Application No. 63/388,158, filed Jul. 11, 2022; 63/423,267, filed Nov. 7, 2022; 63/452,590, filed Mar. 16, 2023; 63/469,680, filed May 30, 2023; and 63/469,924, filed May 31, 2023, which are incorporated by reference as if disclosed herein in their entireties. This application also claims the benefit of U.S. Provisional Application No. 63/715,364, Nov. 1, 2024, and 63/547,242, filed Nov. 3, 2023, which are incorporated by reference as if disclosed here in their entireties.

### STATEMENT REGARDING FEDERALLY SPONSORED RESEARCH OR DEVELOPMENT

This invention was made with government support under grant number 70NANB21H085 awarded by the National Institute of Standards and Technology (NIST). The government has certain rights in the invention.

### INCORPORATION OF SEQUENCE LISTING

The contents of the file named “Sequence_final.xml”, which was created on Nov. 4, 2024 and is 4 KB in size, are hereby incorporated by reference in their entireties.

### BACKGROUND

Today, many mRNA products are being developed at companies such as Moderna, Pfizer, BioNTech and Greenlight Biosciences for treatment of many diseases. In a particular example, continuous production and purification would speed-up manufacturing and reduce costs of production of mRNA vaccine products. Replacing chromatographic purification methods, which can be slow, diffusive, and disruptive, with alternatives could provide myriad benefits towards this continuous production. However efforts to perform this replacement and thereby enhance the possibility of designing and testing a continuous purification process are not known. While commercial affinity chromatography and affinity polymer monoliths are alternate technologies, neither are conducive to continuous operation and both suffer from pressure drop and mass transfer limitations (with >t_{R} detrimental to mRNA stability).

By way of example, affinity columns exhibit pressure drop, mass transfer, and kinetic limitations that result in decreased bed depth due to high pressure drops, increased bed diameters, the need for multiple beds in parallel with rigid and smaller particles, and relatively long residence times, i.e., low Peclet number,

$$P e = \frac{( {L}_{D}^{2} / D )}{( {V}_{0} / Q )} ≪ 1 = {t}_{D} / {t}_{R}$$

where t_{D}=diffusion time. Chromatographic separations do not tolerate large sticky molecules like mRNA and need large footprints (many bead volumes).

What is desired, therefore, are systems for the fast, efficient, and continuous purification of an mRNA target from a product solution, that is also gentle enough so that functionality of the recovered mRNA is retained.

### SUMMARY

Aspects of the present disclosure are directed to an oligomeric ligand including a linker construct, one or more oligomer constructs, and one or more spacer constructs positioned between the linker construct and the one or more oligomer constructs. In some embodiments, the oligomer constructs include between 5% and about 10% guanine and about 90% to about 95% thymine. In some embodiments, the linker construct includes an acrylate group, an azide group, or combinations thereof. In some embodiments, the one or more spacer constructs include a polyethyleneglycol (PEG) group, a hydrocarbyl group, or combinations thereof. In some embodiments, the one or more spacer constructs includes a three-carbon group (C3), a hexa-ethyleneglycol group (18AHS), or combinations thereof. In some embodiments, the oligomer constructs include between about 20 and about 60 nucleotides including at least 15thymines and at least 1 guanine. In some embodiments, the oligomeric ligand includes a chemical structure including Acryl-18AHS-18AHS-TTT TTT TTT GTT TTT TTT TT. In some embodiments, the oligomeric ligand includes a chemical structure including Acryl-C3-C3-C3-C3-C3-C3-TTT TTT TTT GTT TTT TTT TT. In some embodiments, the oligomeric ligand includes a chemical structure including Acryl-18AHS-18AHS-TTT TTT TTT GTT TTT TTT TGT TTT TTT TTG TTT TTT TTT GTT TTT TTT TGT TTT TTT TTT. In some embodiments, the oligomeric ligand includes a chemical structure including Acryl-C3-C3-C3-C3-C3-C3-TTT TTT TTT GTT TTT TTT TGT TTT TTT TTG TTT TTT TTT GTT TTT TTT TGT TTT TTT TTT. In some embodiments, the oligomeric ligand includes a chemical structure including (N₃)-PEG-PEG-PEG-PEG-PEG-TTT TTT TTT GTT TTT TTT TT. In some embodiments, the oligomeric ligand includes a chemical structure including (N₃)-PEG-PEG-PEG-PEG-PEG-TTT TTT TTT GTT TTT TTT TGT TTT TTT TTG TTT TTT TTT GTT TTT TTT TGT TTT TTT TTT.

Aspects of the present disclosure are directed to a method for modifying a surface for purification of mRNA, including: providing a substrate having a surface; and grafting a plurality of ligands on the surface to form a ligand layer. In some embodiments, the ligand layer includes a linker bound to the surface and one or more oligomer constructs bound to the linker. In some embodiments, the oligomer constructs include between 5% and about 10% guanine and about 90% to about 95% thymine.

In some embodiments, grafting a plurality of ligands on the surface to form a ligand layer includes grafting an initiator to hydroxyl groups on the surface; and grafting the plurality of ligands to the initiators. In some embodiments, the linker includes one or more spacer constructs, the one or more spacer constructs including a polyethyleneglycol (PEG) group, a hydrocarbyl group, or combinations thereof. In some embodiments, the one or more oligomer constructs includes between about 20 and about 60 nucleotides including at least 15 thymines and at least 1 guanine.

In some embodiments, grafting the plurality of ligands to the initiators includes contacting the initiator with the plurality of ligands in a reaction medium including PMDETA (N,N,N′,N″,N″-Pentamethyldiethylenetriamine) and copper catalyst, wherein the linker includes an acrylate group. In some embodiments, grafting the plurality of ligands to the initiators includes contacting the initiator in a reaction medium including PMDETA and dibenzo cyclooctyne (strained alkyne)(DBCO)-PEG-acrylate to form a modified surface; and contacting the ligand with the modified surface, wherein the linker includes an azide group. In some embodiments, the ligands on the surface include a chemical structure including Acryl-18AHS-18AHS-TTT TTT TTT GTT TTT TTT TT; Acryl-C3-C3-C3-C3-C3-C3-TTT TTT TTT GTT TTT TTT TT; Acryl-18AHS-18AHS-TTT TTT TTT GTT TTT TTT TGT TTT TTT TTG TTT TTT TTT GTT TTT TTT TGT TTT TTT TTT; Acryl-C3-C3-C3-C3-C3-C3-TTT TTT TTT GTT TTT TTT TGT TTT TTT TTG TTT TTT TTT GTT TTT TTT TGT TTT TTT TTT; (N₃)-PEG-PEG-PEG-PEG-PEG-TTT TTT TTT GTT TTT TTT TT; (N₃)-PEG-PEG-PEG-PEG-PEG-TTT TTT TTT GTT TTT TTT TGT TTT TTT TTG TTT TTT TTT GTT TTT TTT TGT TTT TTT TTT; or combinations thereof.

Aspects of the present disclosure are directed to a modified substrate for purification of mRNA including a surface; and a ligand layer on the surface. In some embodiments, the ligand layer includes a plurality of ligands includes a linker construct bound to the surface, the linker construct including an acrylate group, an azide group, or combinations thereof. In some embodiments, the ligand layer includes one or more spacer constructs bound to the linker construct, wherein the one or more spacer constructs includes a polyethyleneglycol (PEG) group, a hydrocarbyl group, or combinations thereof. In some embodiments, the ligand layer includes one or more oligomer constructs bound to at least one spacer construct. In some embodiments, the oligomer constructs include between 5% and about 10% guanine and about 90% to about 95% thymine, and between about 20 and about 60 nucleotides including at least 15 thymines and at least 1 guanine. In some embodiments, the surface is composed of poly (aryl sulfone), cellulose, silica, or combinations thereof. In some embodiments, the ligands include a chemical structure including Acryl-18AHS-18AHS-TTT TTT TTT GTT TTT TTT TT; Acryl-C3-C3-C3-C3-C3-C3-TTT TTT TTT GTT TTT TTT TT; Acryl-18AHS-18AHS-TTT TTT TTT GTT TTT TTT TGT TTT TTT TTG TTT TTT TTT GTT TTT TTT TGT TTT TTT TTT; Acryl-C3-C3-C3-C3-C3-C3-TTT TTT TTT GTT TTT TTT TGT TTT TTT TTG TTT TTT TTT GTT TTT TTT TGT TTT TTT TTT; (N₃)-PEG-PEG-PEG-PEG-PEG-TTT TTT TTT GTT TTT TTT TT; (N₃) -PEG-PEG-PEG-PEG-PEG-TTTTTT TTT GTT TTT TTT TGT TTT TTT TTG TTT TTT TTT GTT TTT TTT TGT TTT TTT TTT; or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chemical structure of an oligomeric ligand according to some embodiments of the present disclosure;

FIG. 2 is a schematic representation of a modified substrate according to some embodiments of the present disclosure;

FIG. 3A is a chart of a method for modifying a surface and/or substrate according to some embodiments of the present disclosure;

FIG. 3B is a chart of a method for modifying a surface and/or substrate according to some embodiments of the present disclosure;

FIG. 3C is a chart of a method for modifying a surface and/or substrate according to some embodiments of the present disclosure;

FIG. 4 is a flowchart showing modification of a surface and/or substrate according to some embodiments of the present disclosure;

FIG. 5 is a graph showing the effect of catalyst surface exposure during modification of a surface and/or substrate according to some embodiments of the present disclosure;

FIG. 6A is a graph showing x-ray photoelectron spectroscopy (XPS) measurements showing initiator concentration during modification of a surface and/or substrate according to some embodiments of the present disclosure;

FIG. 6B is a graph showing ligand grafting on initiator modified surfaces and/or substrates; and

FIG. 7 is a flowchart showing modification of a surface and/or substrate according to some embodiments of the present disclosure.

### DETAILED DESCRIPTION

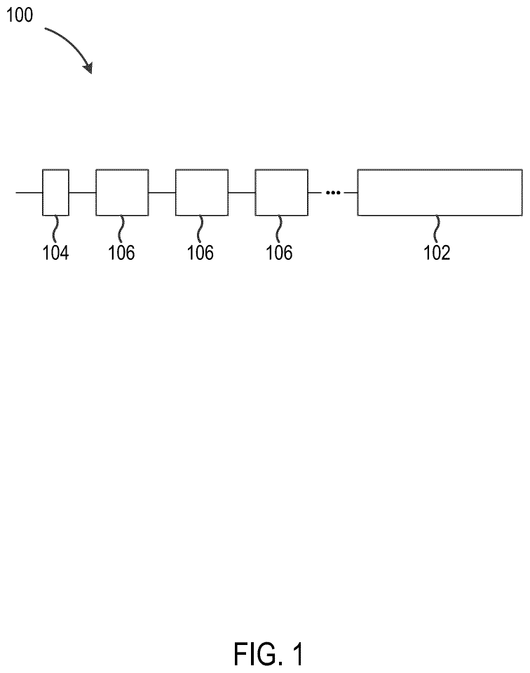

Referring now to FIG. 1, some embodiments of the present disclosure are directed to an oligomeric ligand 100. In some embodiments, oligomeric ligand 100 includes an oligomer construct 102. In some embodiments, oligomeric ligand 100 includes a plurality of oligomer constructs 102. In some embodiments, oligomer construct 102 is configured to bind oligo-dA regions on target constructs, e.g., dAₙ tails on mRNA molecules, including mRNA vaccines against coronaviruses. In some embodiments, oligomer construct 102 is configured to bind oligo-dAₙ regions wherein n is greater than about 20, 30, 40, 50, 60, 70, 80, 90, 100, 110, 120, 130, 140, 150, etc., or combinations thereof. In some embodiments, oligomer construct 102 is configured to reversibly bind the oligo-dA regions. In some embodiments, oligomer constructs 102 include between 5% and about 10% guanine and about 90% to about 95% thymine. In some embodiments, oligomer constructs 102 include between about 20 and about 60 nucleotides including at least 15 thymines and at least 1 guanine. In some embodiments, oligomer constructs 102 include a plurality of nucleotide repeats. In some embodiments, the nucleotide repeats include between about 5 and about 15 nucleotides. In some embodiments, the nucleotide repeats include about 10 nucleotides. In some embodiments, the nucleotide repeats include thymine and guanine residues. In some embodiments, the nucleotide repeats include only thymine and guanine residues. In some embodiments, oligomer constructs 102 include only thymine and guanine residues In some embodiments, oligomer constructs 102 include between about 20 and about 60 nucleotides including only thymine and guanine residues. In some embodiments, oligomer constructs 102 include SEQ. ID NO.: 1, SEQ. ID NO.: 2, or combinations thereof.

In some embodiments, oligomeric ligand 100 includes a linker construct 104. In some embodiments, oligomeric ligand 100 includes a plurality of linker constructs 104. In some embodiments, linker construct 104 includes one or more structures for binding to a surface, e.g., of a membrane. In some embodiments, linker construct 104 includes one or more structures for reversibly binding to the surface. In some embodiments, linker construct includes one or more reactive chemical groups for forming a bond with one or more chemical groups on the surface. In some embodiments, the reactive chemical groups include an acrylate group, an azide group, or combinations thereof.

In some embodiments, oligomeric ligand 100 includes one or more spacer constructs 106 positioned between one or more linker constructs 104 and one or more oligomer constructs 102. In some embodiments, oligomeric ligand 100 includes a plurality of spacer constructs 106 positioned between one or more linker constructs 104 and one or more oligomer constructs 102. In some embodiments, spacer constructs 106 include a polyethyleneglycol group, a hydrocarbyl group, or combinations thereof. As used herein, the term “hydrocarbyl” refers to hydrocarbon compounds that are saturated or unsaturated, chain or branched, substituted or unsubstituted, or combinations thereof. In some embodiments, spacer constructs 106 include a three-carbon group (C3), a hexa-ethyleneglycol group (18AHS), or combinations thereof.

In some embodiments, oligomeric ligand 100 includes a chemical structure including Acryl-18AHS-18AHS-TTT TTT TTT GTT TTT TTT TT; Acryl-C3-C3-C3-C3-C3-C3-TTT TTT TTT GTT TTT TTT TT; Acryl-18AHS-18AHS-TTT TTT TTT GTT TTT TTT TGT TTT TTT TTG TTT TTT TTT GTT TTT TTT TGT TTT TTT TTT; Acryl-C3-C3-C3-C3-C3-C3-TTT TTT TTT GTT TTT TTT TGT TTT TTT TTG TTT TTT TTT GTT TTT TTT TGT TTT TTT TTT; (N₃)-PEG-PEG-PEG-PEG-PEG-TTT TTT TTT GTT TTT TTT TT; (N₃)-PEG-PEG-PEG-PEG-PEG-TTT TTT TTT GTT TTT TTT TGT TTT TTT TTG TTT TTT TTT GTT TTT TTT TGT TTT TTT TTT, or combinations thereof.

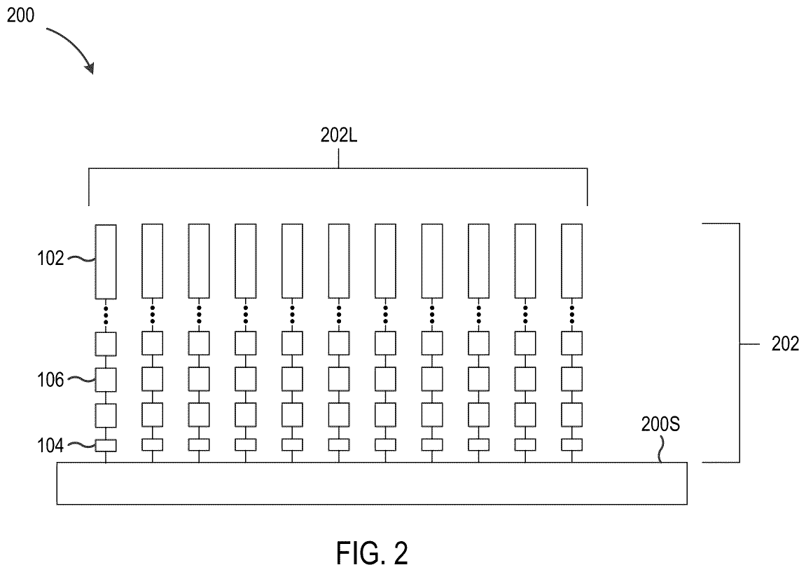

Referring now to FIG. 2, some embodiments of the present disclosure are directed to a modified substrate 200 for purification of a target from a solution. In some embodiments, the target is single stranded ribonucleic acids (ssRNA), e.g., oligo-dA tails from mRNA, including mRNA vaccine molecules. In some embodiments, the mRNA vaccine is for use against coronaviruses, e.g., SARS-CoV-2. In some embodiments, the substrate is a separation medium, e.g., a porous or semi-porous membrane, non-porous substrate, bead, etc., or combinations thereof. In some embodiments, substrate 200 includes a surface 200S. In some embodiments, surface 200S is composed of poly (aryl sulfone), cellulose, silica, or combinations thereof.

In some embodiments, substrate 200 includes one or more ligand layers 202 on surface 200S. In some embodiments, ligand layer 202 includes a plurality of ligands 202L. In some embodiments, ligands 202L are configured to bind the target. In some embodiments, ligands 202L are configured to reversibly bind the target. In some embodiments, ligands 202L are configured to be contacted with the solution and bind the target such that the solution can be removed and the target remains bound to surface 200S. In some embodiments, each of the ligands in plurality of ligands 202L are substantially the same, i.e., are configured to bind the same target. In some embodiments, plurality of ligands 202L includes two or more different ligands, i.e., include ligands configured to bind different targets.

As discussed above, in some embodiments, ligands 202L include one or more linker constructs, e.g., linker constructs 104. In some embodiments, ligands 202L are bound to surface 200S via the linker constructs. In some embodiments, the linker constructs include an acrylate group, an azide group, or combinations thereof.

In some embodiments, ligands 202L include one or more spacer constructs, e.g., spacer constructs 106, bound to linker constructs. In some embodiments, ligands 202L include a plurality of spacer constructs bound to linker constructs. In some embodiments, the spacer constructs include a polyethyleneglycol group, a hydrocarbyl group, or combinations thereof. In some embodiments, the spacer constructs include C3, 18AHS, or combinations thereof.

In some embodiments, ligands 202L include one or more oligomer constructs, e.g., oligomer constructs 102, bound to at least one spacer construct. As discussed above, in some embodiments, the oligomer constructs include between 5% and about 10% guanine and about 90% to about 95% thymine. In some embodiments, the oligomer constructs include between about 20 and about 60 nucleotides including at least 15 thymines and at least 1 guanine. In some embodiments, the oligomer constructs include a plurality of nucleotide repeats. In some embodiments, the nucleotide repeats include between about 5 and about 15 nucleotides. In some embodiments, the nucleotide repeats include about 10 nucleotides. In some embodiments, the nucleotide repeats include thymine and guanine residues. In some embodiments, the nucleotide repeats include only thymine and guanine residues. In some embodiments, the oligomer constructs include only thymine and guanine residues In some embodiments, the oligomer constructs include between about 20 and about 60 nucleotides including only thymine and guanine residues. In some embodiments, the oligomer constructs include SEQ. ID NO.: 1, SEQ. ID NO.: 2, or combinations thereof. Upon contact of a solution containing the oligo-dA target with the modified substrate, the oligo-dA target binds to ligands 202L, and the solution can be subsequently removed. The oligo-dA target can then be recovered from the ligand layer 202, thus achieving separation from the solution and other components therein and purification of the target.

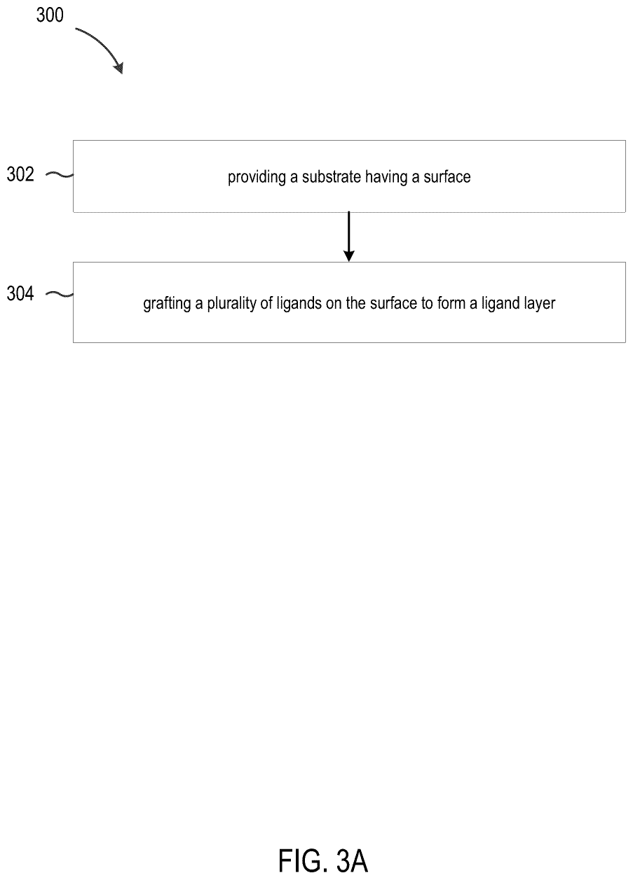

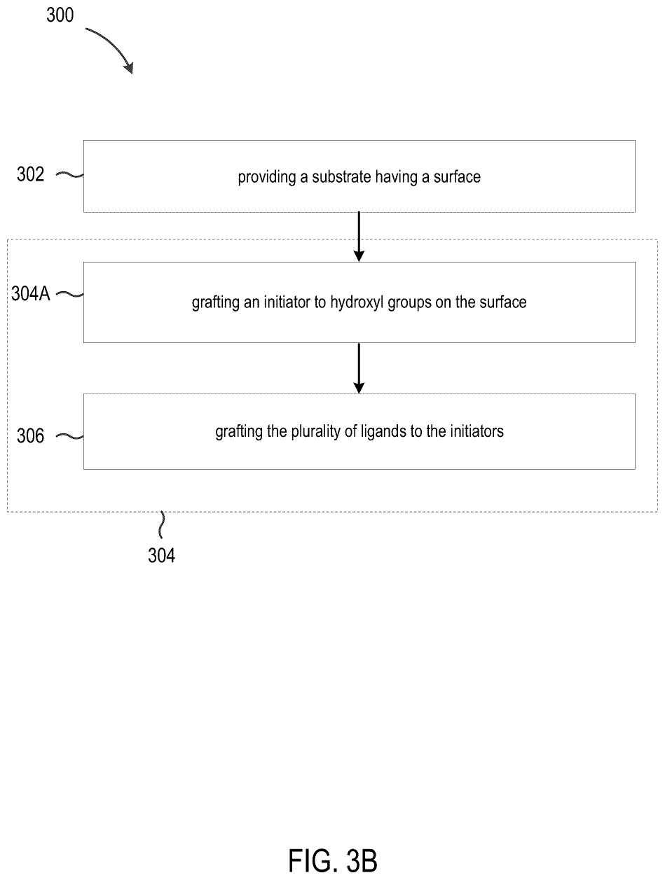

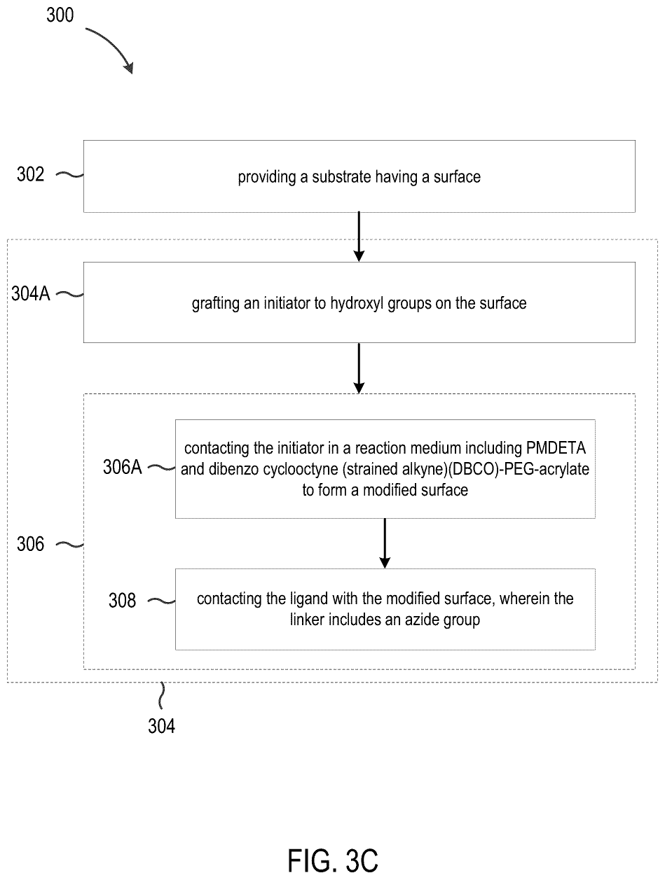

Referring now to FIG. 3A, some embodiments of the present disclosure are directed to a method 300 for modifying a surface for purification of a target, e.g., mRNA, from a solution. In some embodiments, at 302, a substrate having a surface is provided. As discussed above, in some embodiments, the surface is composed of, or is made to be composed of, poly(aryl sulfone), cellulose, silica, or combinations thereof. At 304, a plurality of ligands is grafted on the surface to form a ligand layer. As discussed above, in some embodiments, the ligand layer includes a linker bound to the surface and one or more oligomer constructs bound to the linker. In some embodiments, the linker includes one or more spacer constructs, e.g., a polyethyleneglycol group, a hydrocarbyl group, or combinations thereof. In some embodiments, the linker includes one or more linker constructs. In some embodiments, the linker constructs include an acrylate group, an azide group, or combinations thereof.

In some embodiments, the oligomer constructs include between 5% and about 10% guanine and about 90% to about 95% thymine. In some embodiments, the oligomer constructs include between about 20 and about 60 nucleotides including at least 15 thymines and at least 1 guanine. In some embodiments, the oligomer constructs include a plurality of nucleotide repeats. In some embodiments, the nucleotide repeats include between about 5 and about 15 nucleotides. In some embodiments, the nucleotide repeats include about 10 nucleotides. In some embodiments, the nucleotide repeats include thymine and guanine residues. In some embodiments, the nucleotide repeats include only thymine and guanine residues. In some embodiments, the oligomer constructs include only thymine and guanine residues In some embodiments, the oligomer constructs include between about 20 and about 60 nucleotides including only thymine and guanine residues. In some embodiments, the oligomer constructs include SEQ. ID NO.: 1, SEQ. ID NO.: 2, or combinations thereof.

In some embodiments, grafting 304 is performed in the presence of one or more solvents. In some embodiments, the solvent is any suitable solvent for use with the particular modified surface and ligands, e.g., water, methanol, etc., or combinations thereof. In some embodiments, the volumetric ratio of solvent to monomer, e.g., acrylate ligand, in step 304 is less than about 10:1. In some embodiments, In some embodiments, the volumetric ratio of solvent to monomer in step 304 is between about 2:1 and about 6:1. In some embodiments, the volumetric ratio of solvent to monomer in step 304 is about 5:1. In some embodiments, the volumetric ratio of solvent to monomer in step 304 is about 6:1.

Referring now to FIG. 3B, in some embodiments, grafting 304 a plurality of ligands on the surface to form a ligand layer includes grafting an initiator 304A to hydroxyl groups on the surface. At 306, the plurality of ligands is then grafted to the initiators. In some embodiments, grafting 306 the plurality of ligands to the initiators includes contacting the initiator with the plurality of ligands in a reaction medium including PMDETA (N,N,N′,N″,N″-Pentamethyldiethylenetriamine) and copper catalyst.

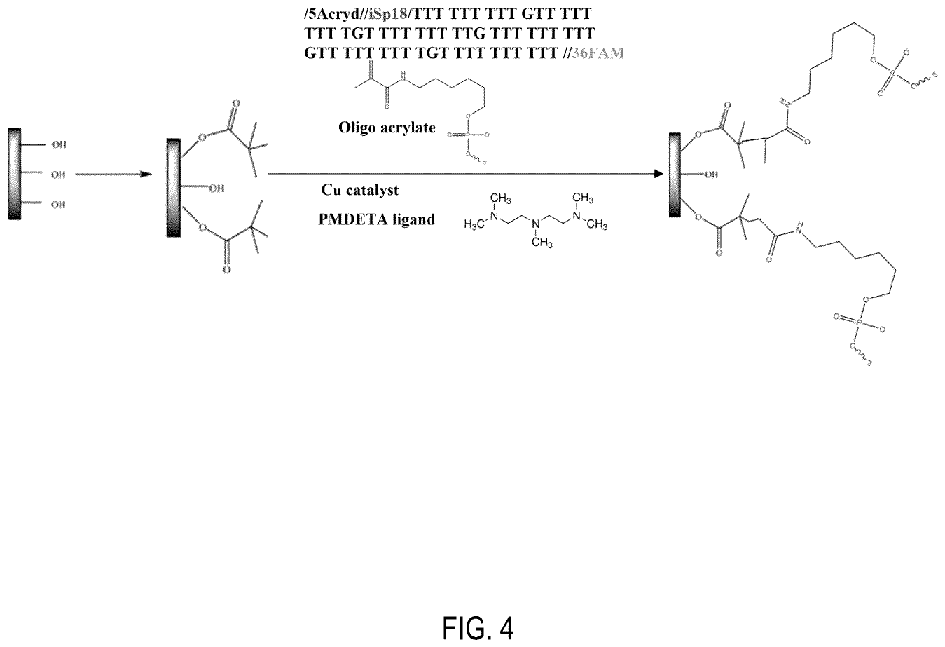

Referring now to FIG. 4, an exemplary reaction flowchart consistent with embodiments of method 300 is shown. In this exemplary embodiment, an initiator, BIBB (α-Bromoisobutyryl bromide), is grafted to a surface of regenerated cellulose (RC) via hydroxyl groups on the RC. As discussed above, the present disclosure is not intended to be limited to this particular surface composition, as the hydroxyl groups could also be natively or engineered to be present on the surfaces of membranes, beads, etc. with alternative compositions. Single-Electron Transfer-Living Radical Polymerization (SET-LRP) is then used in the presence of the plurality of ligands with PMDETA, which forms a complex with available Cu(I) catalyst to increase kinetic rate constant values. Acrylate groups in the ligands bind to the BBIB-modified surface, with the oligomeric constructs separated and extending away from the surface via the spacer constructs.

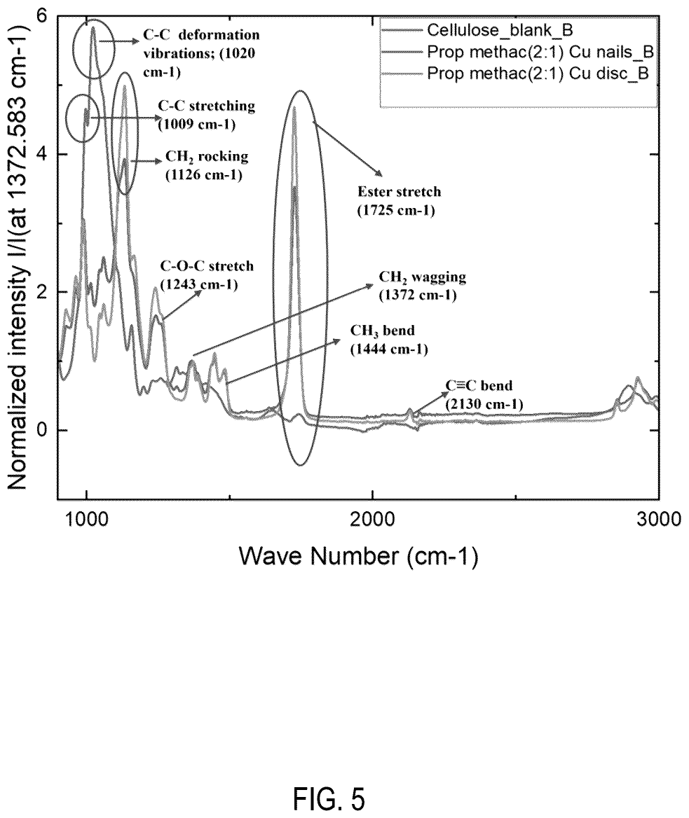

Referring now to FIG. 5, in some embodiments, a single solid plate copper (Cu0) plate catalyst was used for the SET-LRP reaction. The plate catalyst was covered and pressed onto the substrate surface to allow for even copper accessibility to the surface, e.g., into the pores of a membrane that results in even reaction conditions throughout the membrane. Gaps between the copper plate and the pores can result in uneven reaction conditions. The complete contact of the surface to be modified with the surface of the solid Cu catalyst improved grafting density on the surface for a given initial solvent: monomer ratio. With intermittent contact, the degree of grafting is reduced.

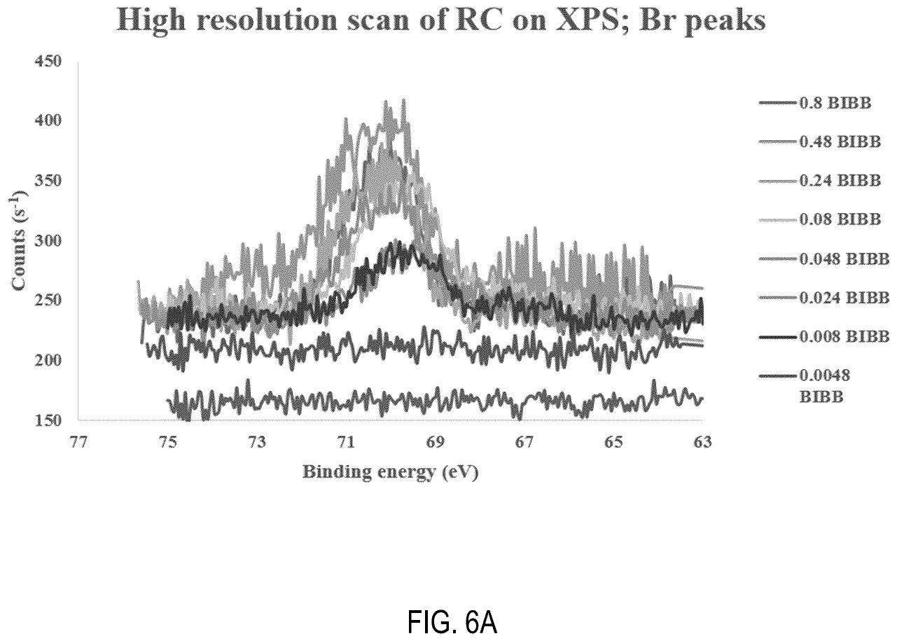

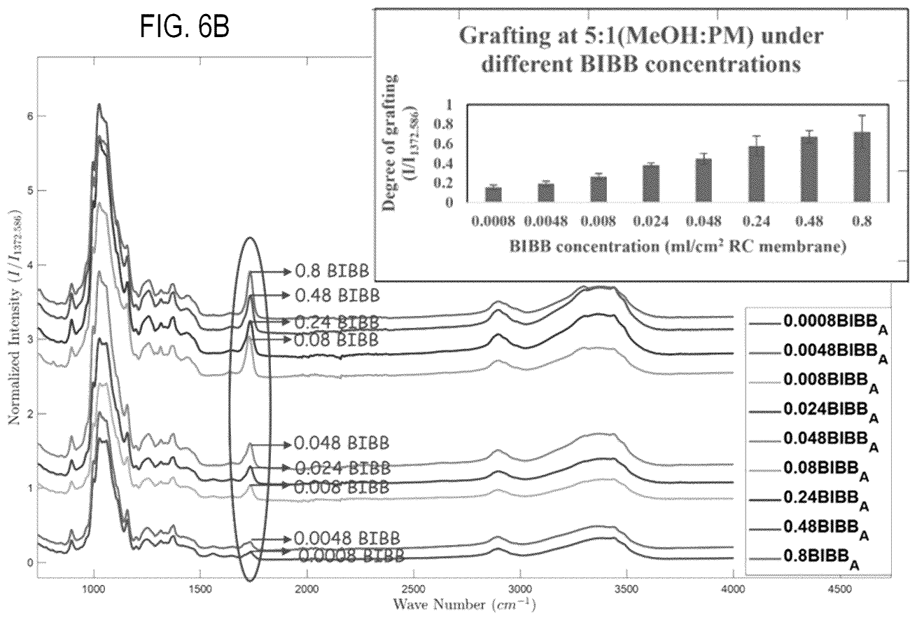

Referring now to FIGS. 6A-6B, without wishing to be bound by theory, the effect of the density of free radical initiation sites (BIBB) were dependent on the amount of initiator. In some embodiments, as observed with x-ray photoelectron spectroscopy (XPS) measurements, the bromine free radical initiation sites were at 0.48 ml BIBB/cm² of membrane (see FIG. 6A). The intensity of the ester peak after grafting of the acrylate monomer by SET-LRP increased linearly with increasing amounts of BIBB addition (see FIG. 6B).

Referring now to FIG. 3C, in some embodiments, grafting 306 the plurality of ligands to the initiators includes contacting the initiator 306A in a reaction medium including PMDETA and dibenzo cyclooctyne (strained alkyne)(DBCO)-PEG-acrylate to modify the surface. At 308, a plurality of ligands are contacted with the modified surface to bind the ligands thereon.

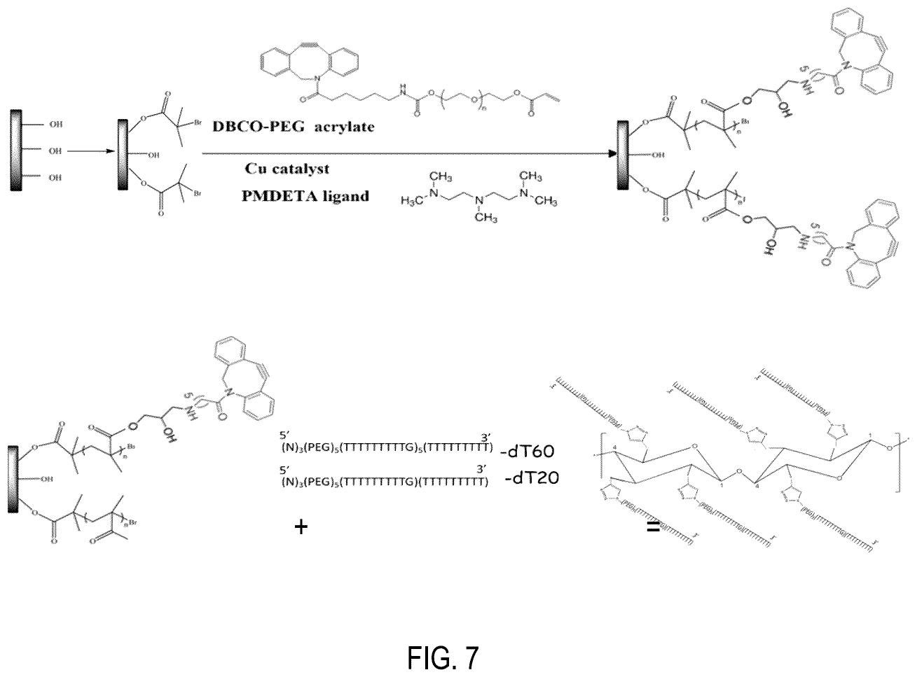

Referring now to FIG. 7, another exemplary reaction flowchart consistent with embodiments of method 300 from FIG. 3C is shown. In this exemplary embodiment, despite being more time consuming, multiple levels of control of grafting density are exhibited, e.g., at the BIBB modification stage, at the SET-LRP grafting stage, and at the strain promoted azide alkyne click (SPAAC) reaction stage, for efficient manufacture of modified surfaces with defined grafting densities based on the application target. There is no scope for residual Cu contamination on the final membranes since all the Cu can be washed off after Step 1. Also this is an economical option in terms of oligonucleotide usage since a small percentage of the starting amount of the oligonucleotide will get grafted onto the surface of the membrane by SET-LRP reaction. In comparison, but without wishing to be bound by theory, SPAAC reaction is more efficient and targeted hence similar surface grafting densities can be obtained with lower starting amounts of oligonucleotides.

Systems and methods of the present disclosure are advantageous for providing the increased quality while reducing the time and cost of mRNA vaccine purification, facilitating continuous production thereof. Embodiments of the oligomeric ligands of the present disclosure allow selective affinity capture of a desired mRNA molecule (such as an oligo-dA₂₀ or COVID-19 vaccine mRNA molecule from an in vitro transcription (IVT) reaction mixture) in solution or on a surface. The different binding chemistries used to attach the various oligomeric ligands to a surface provide a tool to generate affinity membranes, porous beads, etc. that can be tuned according to the desired purification. By way of example, SET-LRP can be used to “graft” molecules to a light sensitive polymeric surface like poly (aryl sulfone) or an initiator modified surface like RC. The ligands can be attached to the surface via reactive linkers such as acrylate or azide group, with a plurality of spacers such as PEG and hydrocarbyl groups advantageously providing additional space between the 5′ end of the oligomer constructs and the surface.

Surfaces modified with the oligomeric constructs according to some embodiments of the present disclosure include a mixture of thymine and guanine that exhibit improved binding of mRNA with oligo-dAₙ tails, e.g., for purification of mRNA during production and commercialization. The systems and methods of the present disclosure are better than the standard approach for affinity binding of single stranded nucleic acids, showing a ˜40% increase in binding for both 2 and 24 hours of binding time.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. An oligomeric ligand, comprising:
a linker construct;
one or more oligomer constructs; and
one or more spacer constructs positioned between the linker construct and the one or more oligomer constructs,
wherein the oligomer constructs include between 5% and about 10% guanine and about 90% to about 95% thymine.

2. The oligomeric ligand according to claim 1, wherein the linker construct includes an acrylate group, an azide group, or combinations thereof.

3. The oligomeric ligand according to claim 1, wherein the one or more spacer constructs include a polyethyleneglycol (PEG) group, a hydrocarbyl group, or combinations thereof.

4. The oligomeric ligand according to claim 3, wherein the one or more spacer constructs includes a three-carbon group (C3), a hexa-ethyleneglycol group (18AHS), or combinations thereof.

5. The oligomeric ligand according to claim 1, wherein the oligomer constructs include between about 20 and about 60 nucleotides including at least 15 thymines and at least 1 guanine.

6. The oligomeric ligand according to claim 5, wherein the oligomeric ligand includes a chemical structure including Acryl-18AHS-18AHS-TTT TTT TTT GTT TTT TTT TT.

7. The oligomeric ligand according to claim 5, wherein the oligomeric ligand includes a chemical structure including Acryl-C3-C3-C3-C3-C3-C3-TTT TTT TTT GTT TTT TTT TT.

8. The oligomeric ligand according to claim 5, wherein the oligomeric ligand includes a chemical structure including Acryl-18AHS-18AHS-TTTTTT TTT GTT TTT TTT TGT TTT TTT TTG TTT TTT TTT GTT TTT TTT TGT TTT TTT TTT.

9. The modified substrate according to claim 5, wherein the oligomeric ligand includes a chemical structure including Acryl-C3-C3-C3-C3-C3-C3-TTT TTT TTT GTT TTT TTT TGT TTT TTT TTG TTT TTT TTT GTT TTT TTT TGT TTT TTT TTT.

10. The modified substrate according to claim 5, wherein the oligomeric ligand includes a chemical structure including (N₃)-PEG-PEG-PEG-PEG-PEG-TTT TTT TTT GTT TTT TTT TT.

11. The modified substrate according to claim 5, wherein the oligomeric ligand includes a chemical structure including (N₃)-PEG-PEG-PEG-PEG-PEG-TTT TTT TTT GTT TTT TTT TGT TTT TTT TTG TTT TTT TTT GTT TTT TTT TGT TTT TTT TTT.

12. A method for modifying a surface for purification of mRNA, comprising:
providing a substrate having a surface; and
grafting a plurality of ligands on the surface to form a ligand layer, wherein the ligand layer includes:
a linker bound to the surface; and
one or more oligomer constructs bound to the linker,
wherein the oligomer constructs include between 5% and about 10% guanine and about 90% to about 95% thymine.

13. The method according to claim 12, wherein grafting a plurality of ligands on the surface to form a ligand layer comprises:
grafting an initiator to hydroxyl groups on the surface; and
grafting the plurality of ligands to the initiators.

14. The method according to claim 13, wherein:
the linker includes one or more spacer constructs, the one or more spacer constructs including a polyethyleneglycol (PEG) group, a hydrocarbyl group, or combinations thereof; and
the one or more oligomer constructs includes between about 20 and about 60 nucleotides including at least 15 thymines and at least 1 guanine.

15. The method according to claim 14, wherein grafting the plurality of ligands to the initiators further comprises:
contacting the initiator with the plurality of ligands in a reaction medium including PMDETA (N,N,N′,N″,N″-Pentamethyldiethylenetriamine) and copper catalyst, wherein the linker includes an acrylate group.

16. The method according to claim 14, wherein grafting the plurality of ligands to the initiators further comprises:
contacting the initiator in a reaction medium including PMDETA and dibenzo cyclooctyne (strained alkyne)(DBCO)-PEG-acrylate to form a modified surface; and
contacting the ligand with the modified surface, wherein the linker includes an azide group.

17. The method according to claim 14, wherein the ligands on the surface include a chemical structure including:
Acryl-18AHS-18AHS-TTT TTT TTT GTT TTT TTT TT;
Acryl-C3-C3-C3-C3-C3-C3-TTT TTT TTT GTT TTT TTT
TT;
Acryl-18AHS-18AHS-TTT TTT TTT GTT TTT TTT TGT TTT
TTT TTG TTT TTT TTT GTT TTT TTT TGT TTT TTT TTT;
Acryl-C3-C3-C3-C3-C3-C3-TTT TTT TTT GTT TTT TTT
TGT TTT TTT TTG TTT TTT TTT GTT TTT TTT TGT TTT
TTT TTT;
(N₃)-PEG-PEG-PEG-PEG-PEG-TTT TTT TTT GTT TTT TTT
TT;
(N₃)-PEG-PEG-PEG-PEG-PEG-TTT TTT TTT GTT TTT TTT
TGT TTT TTT TTG TTT TTT TTT GTT TTT TTT TGT TTT
TTT TTT;
or combinations thereof.

18. A modified substrate for purification of mRNA, comprising:
a surface; and
a ligand layer on the surface,
wherein the ligand layer includes a plurality of ligands including:
a linker construct bound to the surface, the linker construct including an acrylate group, an azide group, or combinations thereof;
one or more spacer constructs bound to the linker construct, wherein the one or more spacer constructs includes a polyethyleneglycol (PEG) group, a hydrocarbyl group, or combinations thereof; and
one or more oligomer constructs bound to at least one spacer construct,
wherein the oligomer constructs include between 5% and about 10% guanine and about 90% to about 95% thymine, and between about 20 and about 60 nucleotides including at least 15 thymines and at least 1 guanine.

19. The modified substrate according to claim 18, wherein the surface is composed of poly(aryl sulfone), cellulose, silica, or combinations thereof.

20. The modified substrate according to claim 18, wherein the ligands include a chemical structure including:
Acryl-18AHS-18AHS-TTT TTT TTT GTT TTT TTT TT;
Acryl-C3-C3-C3-C3-C3-C3-TTT TTT TTT GTT TTT TTT
TT;
Acryl-18AHS-18AHS-TTT TTT TTT GTT TTT TTT TGT TTT
TTT TTG TTT TTT TTT GTT TTT TTT TGT TTT TTT TTT;
Acryl-C3-C3-C3-C3-C3-C3-TTT TTT TTT GTT TTT TTT
TGT TTT TTT TTG TTT TTT TTT GTT TTT TTT TGT TTT
TTT TTT;
(N₃)-PEG-PEG-PEG-PEG-PEG-TTT TTT TTT GTT TTT TTT
TT;
(N₃)-PEG-PEG-PEG-PEG-PEG-TTT TTT TTT GTT TTT TTT
TGT TTT TTT TTG TTT TTT TTT GTT TTT TTT TGT TTT
TTT TTT;
or combinations thereof.
